(19) United States
(12) Patent Application Publication
(10) Pub. No.: US 2012/0057009 A1
(43) Pub. Date: Mar. 8, 2012

(54) Wireless Endoscope Apparatus

(76) Inventors: Shih-Wen LIAO, Yangmei Township (TW); Ching-Cheng Wu, Yangmei Township (TW); Chih-Liang Fan, Yangmei Township (TW)

(21) Appl. No.: 12/875,253

(22) Filed: Sep. 3, 2010

(51) Int. Cl. A61B 1/04; H04N 7/18

(52) U.S. Cl. 348/76; 600/109; 600/102; 348/E07.085

(57) ABSTRACT

A wireless endoscope apparatus mainly comprises an endoscope main body, a receiving host, a supporting stand and a power connecting wire. The receiving host is electrically connected to the endoscope main body through the power connecting wire. The receiving host can be supported and positioned on an application surface through the supporting stand. Moreover, a captured image is wirelessly transmitted by the signal transmitter of the endoscope main body to the receiving host. The image captured by the image pickup lens then is displayed by a display of the receiving host to avoid the restriction of transmission wires so that fields and explorations, such as medical care, house remodeling, transportation and maintenance, mechanical maintaining and research observation and so on, applied by the invention can be fast and convenient.

### BACKGROUND OF THE INVENTION

1. Field of the Invention

The present invention relates to an endoscope apparatus, and more particularly to a wireless endoscope apparatus that avoids the restriction of transmission wires to further have rapidity and convenience at applications and explorations.

2. Description of the Related Art

A conventional endoscope apparatus is a thin flexible pipe and mainly composed of an image capturing device and a light source. The structure, such as body organs, transportations inside, electronic apparatus's components, and building's crevices, captured by the apparatus can be displayed by a display connected to the flexible pipe after connecting the display so that doctors, architects, electronic engineers, structural engineers and mechanical engineers could diagnose diseases of patients or find problems of detected articles through the image displayed on the display.

Since the technology of the endoscope apparatus is gradually improved, its application field is in widespread use. A conventional endoscope belongs to a wired endoscope apparatus that integrates an image capturing device, a flexible pipe and a display, wherein an optical fiber is contained in the flexible pipe to transmit photoluminescence to another end of the pipe.

While in use, the flexible pipe is put into a portion to be detected, the photoluminescence transmitted from the photoluminescence source can illuminate the portion. The photosensitive element and the image capturing device embedded to the end of the flexible pipe can clearly capture the image of the portion and transmit the signal to the display through the flexible so as to display the image captured by the image capturing device.

However, the conventional wired endoscope apparatus is usually restricted by the length of the flexible pipe. In another word, the flexible pipe may not pass through complicated landforms and complicated articles to cause great restrictions at the applications and distances.

### SUMMARY OF THE INVENTION

In view of the shortcomings of the prior art, the inventor(s) of the present invention based on years of experience in the related industry to conduct extensive researches and experiments, and finally developed a wireless endoscope apparatus.

Therefore, it is an objective of the present invention to overcome the aforementioned shortcoming and deficiency of the prior art by providing a wireless endoscope apparatus that avoids the restriction of transmission wires to further have rapidity and convenience at applications and explorations.

To achieve the foregoing objective, the wireless endoscope apparatus provided by the invention mainly comprises an endoscope main body, a receiving host, a supporting stand and a power connecting wire. The endoscope main body is equipped with an image pickup lens, a signal transmitter, an electric connector and a space for containing a battery. The receiving host has a display, a signal receiver and is stored with a video processing firmware. The receiving host is operated by a built-in battery or an external power. A charging port is connected to the electric connector of the endoscope main body through the power connecting wire to properly charge the battery contained in the endoscope main body. In addition, the supporting stand is pivotally disposed to the receiving host so that the receiving host can be supported and positioned to a utilization surface.

While in use, the image captured from the image pickup lens is wirelessly transmitted to the receiving host through the signal transmitter of the endoscope main body. The image captured by the image pickup lens then is displayed by the display of the receiving host to avoid the restriction of transmission wires so that fields of applications and explorations, such as medical care, house remodeling, transportation and maintenance, mechanical maintaining and research observation and so on, applied by the invention can be fast and convenient.

### BRIEF DESCRIPTION OF THE DRAWINGS

FIG. 1 is a structural schematic diagram of an endoscope main body according to the present invention;

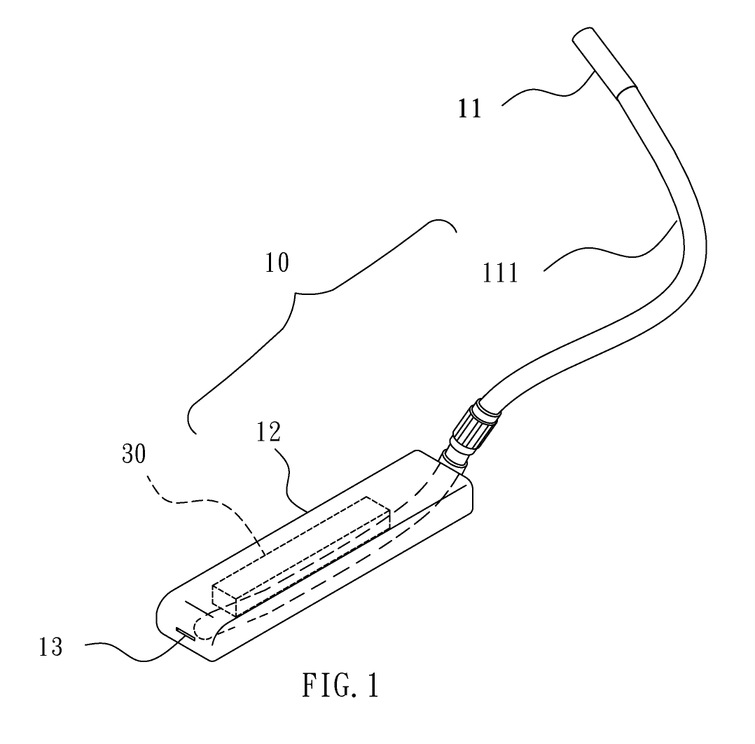

FIG. 2 is a structural drawing of a receiving host according to the present invention;

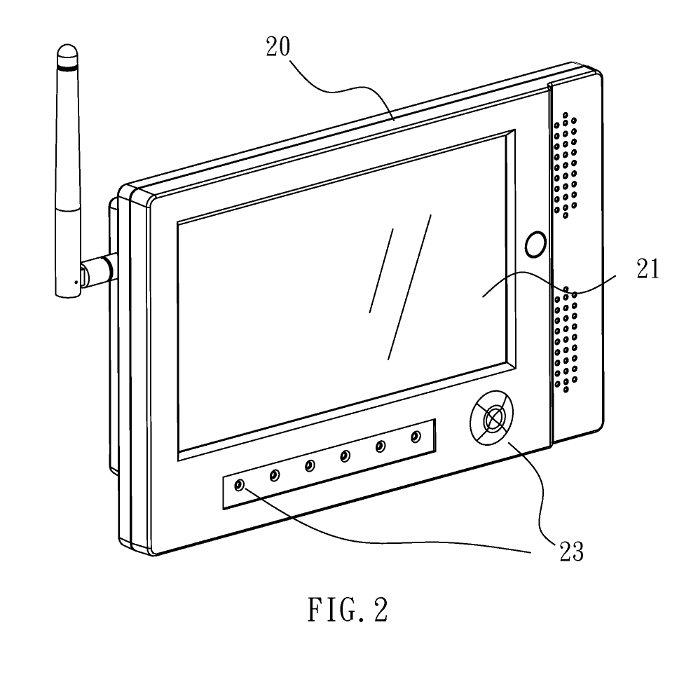

FIG. 3 is a structural schematic diagram of a wireless endoscope apparatus according to a first embodiment of the present invention;

FIG. 4 is a structural schematic diagram of a wireless endoscope apparatus according to a second embodiment of the present invention;

FIG. 5 is a structural decomposition drawing of a wireless endoscope apparatus according to a second embodiment of the present invention; and

FIG. 6 is a functional diagram of a wireless endoscope apparatus according to the present invention.

### DETAILED DESCRIPTION OF THE PREFERRED EMBODIMENTS

The foregoing and other technical characteristics of the present invention will become apparent with the detailed description of the preferred embodiments and the illustration of the related drawings.

A wireless endoscope apparatus of the invention mainly comprises an endoscope main body 10, a receiving host 20, a supporting stand 24 and a power connecting wire 40.

The endoscope main body 10 is equipped with an image pickup lens 11, a signal transmitter 12, an electric connector 13 and a space for containing a battery 30. The image pickup lens 11 has a flexible wire 111 connected to the signal transmitter 12. An end of the flexible wire 111 has a photosensitive element (a charge-coupled device or a complementary metal-oxide-semiconductor) and an optical fiber disposed in the flexible wire 111 to transmit a light source (e.g. white light LED) arranged to the signal transmitter 12 to the end of the flexible wire 111, thereby providing the light source required for the operation of the photosensitive element.

The receiving host 20 is equipped with a display 21 and a signal receiver 26 and a charging port 22 and is stored with a video processing firmware therein.

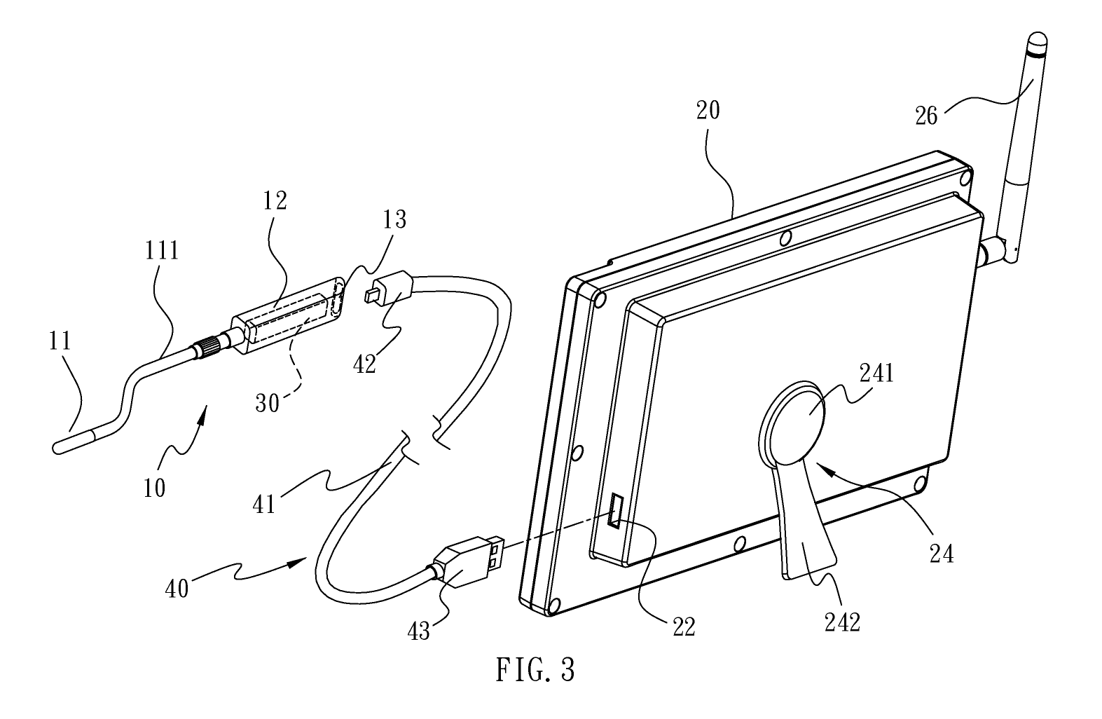

While in implementation, as shown in FIG. 3 for a first embodiment of the invention, the receiving host 20 can be operated with a built-in power or an external power. The battery 30 contained in the endoscope main body 10 can be a rechargeable battery. The endoscope main body 10 has the electric connector 13 electrically connected to the contained battery 30. The power connecting wire 40 is a wire 41. One end of the wire 41 has a first connecting end 42, and another end of the wire 41 has a second connecting end 43. Further, the first connecting end 42 is connected to the electric connector 13. The second connecting end 43 is connected to the charging port 22. The charging port 22 is provided for electrically connecting the electric connector 13 of the endoscope main body 10 through the power connecting wire 40 so that the battery 30 contained in the endoscope main body 10 can be directly charged by the receiving host 20, and the back surface of the receiving host 20 has the supporting stand 24. The supporting stand 24 is composed of a circular pivot portion 241 engaging with a supporting portion 242. The pivot portion 241 is pivotally disposed to the back surface of the display 21 and can be pivoted for movable regulation. In addition, the supporting portion 242 is positioned to the application surface. Actually, the charging port 22 can be a universal serial bus (USB) port, and the charging port 22 can be disposed to a side surface of the receiving host 20.

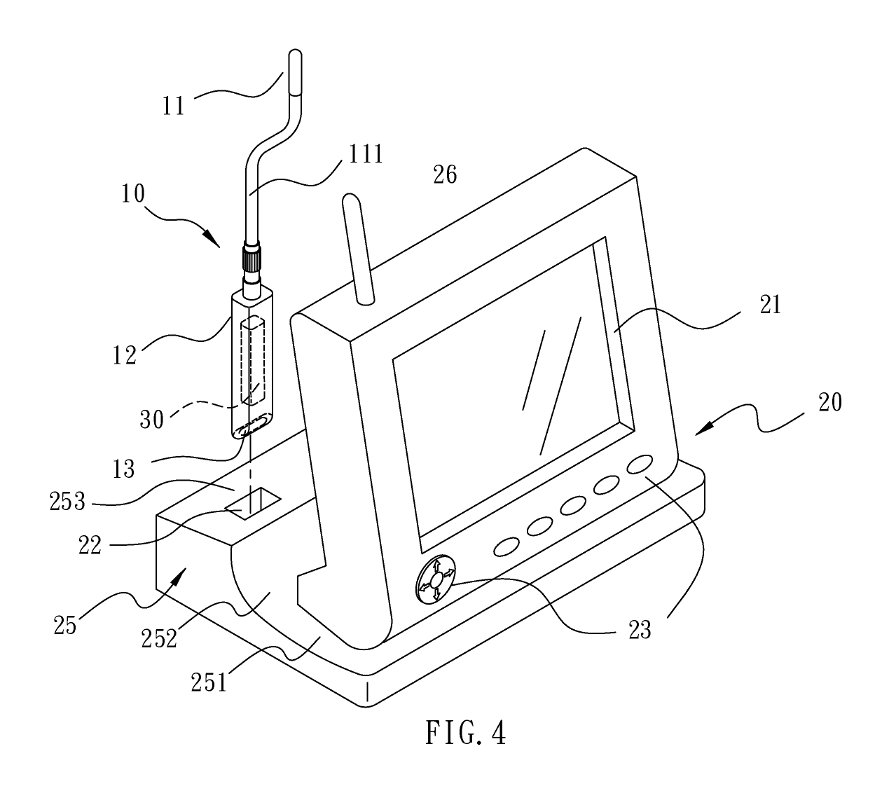

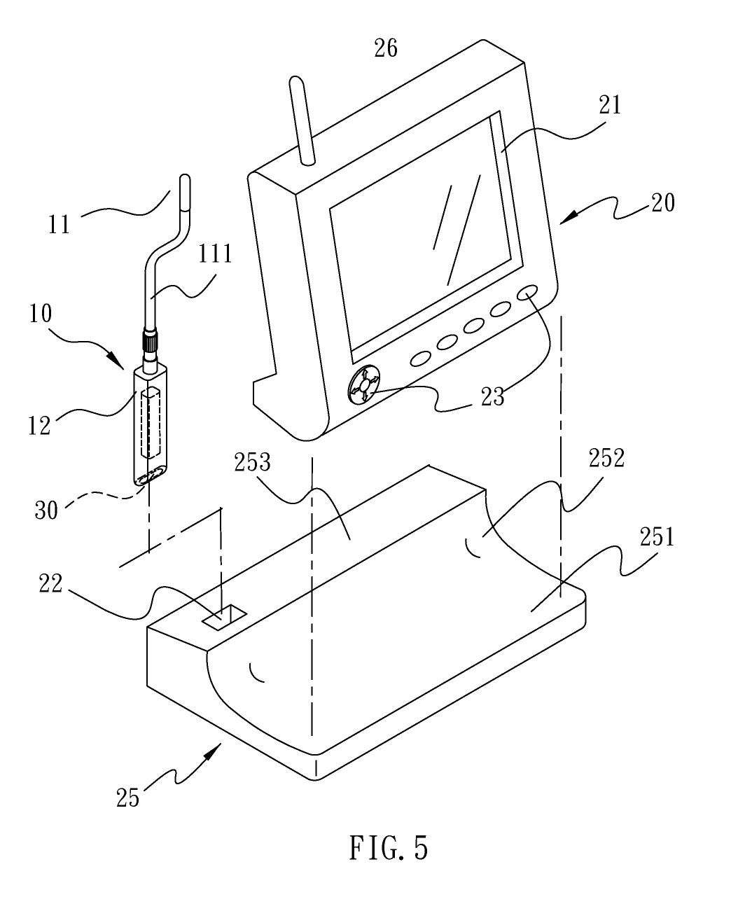

Additionally, as shown in FIG. 4 and FIG. 5 for a second embodiment of the invention, the structure mainly comprises an endoscope main body 10 and a receiving host 20.

The endoscope main body 10 is equipped with an image pickup lens 11, a signal transmitter 12, an electric connector 13 and a space for containing a battery 30. The image pickup lens 11 captures images and wirelessly transmits the images through the signal transmitter 12.

The receiving host 20 is equipped with a display 21, a signal receiver 26 and a base 25 and stores a video processing firmware. The signal receiver 26 receives the signal of the signal transmitter 12, and the images captured by the image pickup lens 11 are displayed by the display 21.

The base is an arc base. The arc base is composed of a platform 253, an arc surface 252 and a flat surface 251 which are extended one by one. The display 21 is fixedly disposed on the flat surface 251, and the platform 253 is provided with a charging port 22. The electric connector 13 of the endoscope main body 10 is electrically connected to the charging port 22. The charging port 22 can be a desk charging port that is directly inserted by the endoscope main body 10 so that the battery 30 contained in the endoscope main body 10 is directly charged by the receiving host 20.

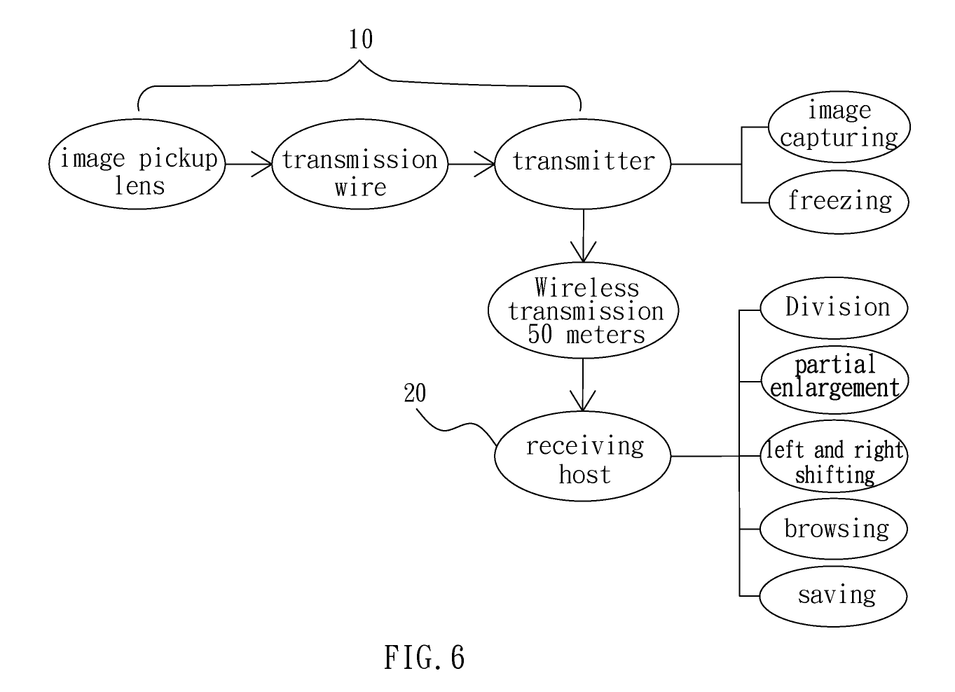

With reference to FIG. 6, when the wireless endoscope apparatus is utilized, the signal transmitter 12 of the endoscope main body 10 wirelessly transmits the images captured by the image pickup lens 11 to the receiving host 20. The images captured by the image pickup lens 11 then are displayed by the display 21 of the receiving host 20. The receiving host 20 can have functions of freezing, dividing, partially enlarging, shifting, browsing the images or storing the images by operating buttons 23 or controlling a touch panel of the apparatus.

Since the images captured by the endo scope main body 10 are wirelessly transmitted to the display 21 of the receiving host 20 at the invention, the restriction of the transmission wires can be avoided so that fields of applications and explorations, such as medical care, house remodeling, transportation and maintenance, mechanical maintaining and research observation and so on, applied by the invention can be fast and convenient.

The wireless endoscope apparatus provided by the present invention improves over the prior art and complies with patent application requirements, and thus is duly filed for patent application. While the invention has been described by device of specific embodiments, numerous modifications and variations could be made thereto by those generally skilled in the art without departing from the scope and spirit of the invention set forth in the claims.

What is claimed is:

1. A wireless endoscope apparatus comprising:
an endoscope main body provided with an image pickup lens, a signal transmitter, an electric connector and a space for containing a battery, the image pickup lens capturing an image that is wirelessly transmitted by the signal transmitter;
a receiving host provided with a display, a signal receiver and a charging port and for storing a video processing firmware therein, the signal receiver for receiving a signal transmitted from the signal transmitter, the display displaying the image captured by the image pickup lens;
a supporting stand composed of a circular pivot portion engaging with a supporting portion, the pivot portion pivotally disposed to a horizontally central point at a back surface of the display and movably pivoted, the supporting portion positioned to an application surface; and
a power connecting wire being a wire, wherein an end of the wire is a first connecting end, and another end of the wire is a second connecting end, and the first connecting end is connected to the electric connector, and the second connecting end is connected to the charging port so that the endoscope main body is electrically connected to the receiving host.

2. The wireless endoscope apparatus as recited in claim 1, wherein the image pickup lens has a flexible wire connected to the signal transmitter, and an end of the flexible wire has a photosensitive element and is provided with an optical fiber therein.

3. The wireless endoscope apparatus as recited in claim 1, wherein the charging port is a universal serial bus port.

4. The wireless endoscope apparatus as recited in claim 1, wherein the receiving host is operated by a built-in battery.

5. The wireless endoscope apparatus as recited in claim 1, wherein the receiving host is operated by an external power.

6. The wireless endoscope apparatus as recited in claim 1, wherein the receiving host has functions of freezing, dividing, partially enlarging, shifting, browsing and storing the image by operating buttons of the apparatus.

7. The wireless endoscope apparatus as recited in claim 1, wherein the receiving host has functions of freezing, dividing, partially enlarging, shifting, browsing and storing the image by controlling a touch panel of the apparatus.

8. A wireless endoscope apparatus comprising:
an endoscope main body equipped with an image pickup lens, a signal transmitter, an electric connector and a space for containing a battery, the image pickup lens capturing an image that is wirelessly transmitted by the signal transmitter; and
a receiving host provided with a display, a signal receiver and a base and for storing a video processing firmware therein, the signal receiver for receiving a signal transmitted from the signal transmitter, the display displaying the image captured by the image pickup lens, the base being an arc base, the arc base composed of a flat surface, an arc surface and a platform which are extended one by one, the platform having a charging port, the display fixedly disposed on the flat surface.

9. The wireless endoscope apparatus as recited in claim 8, wherein the image pickup lens has a flexible wire connected to the signal transmitter, and an end of the flexible wire has a photosensitive element and an optical fiber therein.

10. The wireless endoscope apparatus as recited in claim 8, wherein the charging port is a desk charging port.

11. The wireless endoscope apparatus as recited in claim 8, wherein the receiving host is operated by a built-in battery.

12. The wireless endoscope apparatus as recited in claim 8, wherein the receiving host is operated by an external power.

13. The wireless endoscope apparatus as recited in claim 8, wherein the receiving host has functions of freezing, dividing, partially enlarging, shifting, browsing and storing the image by operating buttons of the apparatus.

14. The wireless endoscope apparatus as recited in claim 8, wherein the receiving host has functions of freezing, dividing, partially enlarging, shifting, browsing and storing the image by controlling a touch panel of the apparatus.
